(19) United States
(12) Patent Application Publication
(10) Pub. No.: US 2022/0155298 A1
(43) Pub. Date: May 19, 2022

(54) METHOD FOR PREDICTING YIELD PERFORMANCE OF A CROP PLANT

(71) Applicant: BASF SE, Ludwigshafen am Rhein (DE)

(72) Inventors: Monika HEILMANN, Limburgerhof (DE); Pilar PUENTE, Limburgerhof (DE); Oliver THIMM, Ludwigshafen (DE); Iain PROCTOR, Limburgerhof (DE); Girish SRINIVAS, Limburgerhof (DE)

(21) Appl. No.: 17/441,240

(22) PCT Filed: Mar. 23, 2020
(86) PCT No.: PCT/EP2020/057905

§ 371 Date: Sep. 20, 2021

(30) Foreign Application Priority Data
Mar. 21, 2019 (EP) 19164363.4
May 23, 2019 (EP) 19176179.0

(51) Int. Cl. G01N 33/569; G16B 20/00; G16B 40/20; G16B 50/10

(52) U.S. Cl. CPC G01N 33/56961; G16B 50/10; G16B 40/20; G16B 20/00

(57) ABSTRACT

The invention relates to a method for predicting yield performance of a crop plant, comprising the steps of receiving metabolite measurements of the crop plant; determining new metabolite features by combining the received metabolite measurements, wherein at least one new metabolite feature is based on a classified average; providing the new metabolite features to a trained machine learning model; and determining yield performance of the crop plant using the provided model. It also relates to a method for training a machine learning model for predicting yield performance of a crop plant; a control unit configured to execute the method for predicting yield performance; to a plant breeding method and a farming method that apply said method; and the use of new metabolite features as determined in said method for prediction of yield performance.

### FIELD OF INVENTION

The present invention relates to a method for predicting yield performance of a crop plant, a method for training a machine learning model for predicting yield performance of a crop plant; a control unit being configured for executing the method for predicting yield performance, a yield evaluation platform, a plant breeding method, and a farming method in which the method for predicting yield performance is applied; and a use of new metabolite features as derived in said method for predicting yield performance of a crop plant.

### BACKGROUND OF THE INVENTION

The world is faced with rapid population growth and expected to reach 9.6 billion by 2050. To meet the growing demand for food, we need to secure our food supply by producing crops with more yield on the same amount of farmland. In addition to protecting crops against pests and disease, growers must also optimize yields and maintain plant health by managing environmental stress while meeting evolving societal expectations of agriculture. Enabling this full yield potential requires solutions beyond conventional crop protection.

For instance abiotic stress for the crop plant due to environmental factors is almost always not preventable during cultivation of the crop plant. However, abiotic stress during the growth of the crop plant, usually leads to loss in yield of the crop plant. Therefore, different variants of crop plants are researched, which comprise an improved resistance to abiotic stress.

In research variables measured such as biomass are not yield predicting and thus not sufficient for enabling decision making.

The chemical and/or biological search space is too large for a high throughput screening in the crop plant of interest for yield or stress tolerance under controlled conditions, for example in a greenhouse. Many candidates with good greenhouse performance fail in the field. Therefore, it is difficult to predict the expected yield of a crop plant candidate. Similar situations on candidate selection or yield prediction occur in farming particularly early in the season, when remote sensing or other techniques fail to produce robust yield predictions.

### SUMMARY OF THE INVENTION

Thus, there is a need for an improved method for predicting yield performance of plants, such as yield loss of a crop plant under certain field conditions. For breeding it is particularly relevant to establish a link between a crop plant response in the field and in the greenhouse. This way the candidate selection process in a greenhouse can be accelerated by selecting those candidates predicted with high yield performance under field conditions in breeding as well as farming scenarios. Moreover, robust predictions on yield performance for a crop plant in early growth stages can be enhanced.

The object of the present invention is solved with the subject matter of the independent claims, wherein further embodiments are incorporated in the dependent claims. It should be noted that the following described aspects and examples of the invention apply equally for the method for predicting yield performance of a crop plant, the control unit, the yield evaluation platform, the plant breeding method, the method for training a machine learning model, and the use of the new metabolite features.

An aspect relates to a method for predicting yield performance of a crop plant, comprising the steps:

receiving metabolite measurements of the crop plant;

determining new metabolite features combining the received metabolite measurements, wherein at least one new metabolite feature is based on a classified average;

providing the new metabolite features to a trained machine learning model; and

determining yield performance of the crop plant using the provided model.

Another aspect relates to a method for training a machine learning model for predicting yield performance of a crop plant comprising the steps:

receiving historical data sets comprising metabolite measurements in connection with a measured yield performance, wherein each data set comprises metabolite measurements for different points in time of the growth cycle for one or more crop plant(s);

determining new metabolite features combining the received historical data sets, wherein at least one new metabolite feature is based on a classified average;

generating a training data set and a test data set based on the historical data sets with new metabolite features;

providing a machine learning model and training the machine learning model based on the training data set;

testing the trained machine learning model based on the test data set.

Advantageously the “classified average” of metabolite measurements adds to the robustness and prediction power of the model, which is with the limited amount of data crucial for valid predictions. Moreover, the proposed approach enables prediction independent of the growth stage (reproductive or vegetative). Hence the methods and systems proposed herein allow to accelerate plant selection or yield forecasts even at an early growth stage. Particularly in early growth stages it is very difficult to provide predictions via standard methods based on remote sensing and the use of metabolites can close such gap.

The term “yield performance”, as used herein, refers to any trait of a crop plant that correlates with yield. The yield performance may be classified based on yield loss or no loss, protein content high or low, biomass high or low, harvested consumable material (e.g. amount of grains, fruits, seeds etc) high or low, or similar classifications. The yield performance may be signified by yield performance prediction data provided by the trained machine learning model.

The terms “crop plant” or “crop”, as used herein, comprises a plant to be cultivated in a crop field and/or a greenhouse. Different variations of crop plants to be analyzed and/or used for further breeding are also described as “crop plant candidates”.

The term “crops includes the following crops: Grain crops, including e.g. cereals (small grain crops) such as wheat (Triticum aestivum) and wheat like crops such as durum (T. durum), einkorn (T. monococcum), emmer (T. dicoccon) and spelt (T. spelta), rye (Secale cereale), triticale (Tritiosecale), barley (Hordeum vulgare); maize (corn; Zea mays); sorghum (e.g. Sorghum bicolour); rice (Oryza spp. such as Oryza sativa and Oryza glaberrima); and sugar cane;

Legumes (Fabaceae), including e.g. soybeans (Glycine max), peanuts (Arachis hypogaea and pulse crops such as peas including Pisum sativum, pigeon pea and cowpea, beans including broad beans (Vicia faba), Vigna spp., and Phaseolus spp. and lentils (lens culinaris var.);

brassicaceae, including e.g. canola (Brassica napus), oilseed rape (OSR, Brassica napus), cabbage (B. oleracea var.), mustard such as B. juncea, B. campestris, B. narinosa, B. nigra and B. tournefortii; and turnip (Brassica rapa var.);
other broadleaf crops including e.g. sunflower, cotton, flax, linseed, sugarbeet, potato and tomato;

TNV-crops (TNV: trees, nuts and vine) including e.g. grapes, citrus, pomefruit, e.g. apple and pear, coffee, pistachio and oilpalm, stonefruit, e.g. peach, almond, walnut, olive, cherry, plum and apricot;

turf, pasture and rangeland;

onion and garlic;

bulb ornamentals such as tulips and narcissus;

conifers and deciduous trees such as pinus, fir, oak, maple, dogwood, hawthorne, crabapple, and rhamnus (buckthorn); and
garden ornamentals such as roses, petunia, marigold and snapdragon.

In one embodiment, the method for controlling undesired vegetation is applied in cultivated rice, maize, pulse crops, cotton, canola, small grain cereals, soybeans, peanut, sugarcane, sunflower, plantation crops, tree crops, nuts or grapes. In another embodiment, the method is applied in cultivated crops selected from glufosinate-tolerant crops.

The methods of the invention are particularly suitable for application in the following crop plants: small grain crops such as wheat, barley, rye, triticale and durum, rice, maize (corn), sugarcane, sorghum, soybean, pulse crops such as pea, bean and lentils, peanut, sunflower, sugarbeet, potato, cotton, brassica crops, such as oilseed rape, canola, mustard, cabbage and turnip, turf, pasture, rangeland, grapes, pomefruit, such as apple and pear, stonefruit, such as peach, almond, walnut, pecans, olive, cherry, plum and apricot, citrus, coffee, pistachio, garden ornamentals, such as roses, petunia, marigold, snap dragon, bulb ornamentals such as tulips and narcissus, conifers and deciduous trees such as pinus, fir, oak, maple, dogwood, hawthorne, crabapple and rhamnus.

The methods of the invention are most suitable for application with the following crop plants: small grain crops such as wheat, barley, rye, triticale and durum, rice, maize, sugarcane, soybean, pulse crops such as pea, bean and lentils, peanut, sunflower, cotton, brassica crops, such as oilseed rape, canola, turf, pasture, rangeland, grapes, stonefruit, such as peach, almond, walnut, pecans, olive, cherry, plum and apricot, citrus and pistachio, especially maize.

The term “classified average”, as used herein, relates to an average of classified values. Such average may be a mean, a median or a weighted average based on weighting parameter(s) or a weighting function. In the context of the present disclosure a classified average may relate to an average of metabolite measurements grouped in a class. The metabolite measurements grouped in a class may relate e.g. to a common chemical or biochemical property. Such measurements may result from different times during the growth stage of a plant and/or from different plants.

The term “model”, as used herein, comprises a mathematical model representing a plant, in particular a crop plant. In this context the model is trained or parametrized based on plant specific features, such as metabolite features derived from metabolite measurements and yield performance.

The term “crop cycle”, as used herein, comprises the growth cycle of a crop plant including different growth stages such as a vegetative growth stage and a reproductive growth stage.

The term “biomarker”, as used herein, comprises a measurable indicator of a biological state or condition of a plant, in particular of a crop plant. While a measured metabolite feature itself does not automatically qualify as a biomarker, the determined new metabolite feature does qualify as a biomarker in the sense that the new metabolite features are prognostic for yield performance.

The term “greenhouse”, as used herein, relates to an indoor facility for cultivating crop plants, preferably a controlled high throughput testing facility for crop plants. In contrast the term “field”, as used herein, relates to an outdoor facility for cultivating crop plants under natural environmental conditions.

Preferably, the received metabolite measurements and/or the new metabolite features are measurable in crop plants within the greenhouse or are measurable in crop plants on a field, e.g. via samples that are provided to a remote testing facility.

The metabolite measurements are combined to new metabolite features. The process of combining the metabolite measurements is preferably achieved by combining the metabolite measurements to a general class and separating those classes again to more specific classes, thereby obtaining the new metabolite feature. Such a system provides a rule-based classification of values and may be referred to as ontology. The generalization is typically based on chemical or biochemical generalizations of metabolites. Accordingly, the term “ontology” as used herein typically relates to a chemical or biochemical generalization of metabolites.

Ontologies may for instance refer to amino acids as general class with different amino acids as sub-classes. Metabolite measurements for one or more plant(s) may be classified in the amino acid ontology and values averaged.

In one embodiment the classified average is determined by the steps of

a) assigning the received metabolite measurements (M) to at least one ontology (F1, F2) and

b) determining the average of the metabolite measurements (M) that are assigned to the same ontology. Hence the metabolite measurements may be classified according to at least one ontology and metabolite measurements classified in the at least one ontology are averaged. Such average may be based on the group of values in each ontology or ontology level. The ontology may include metabolite measurements at different points in time during the crop cycle, such as the vegetative and/or reproductive stage of the crop plant.

In a further embodiment the received metabolite measurements are assigned to different ontologies and/or based on a ratio between product metabolite measurements and substrate metabolite measurements. Following the assignment, measurements grouped in the different ontologies may be averaged per ontology level. Ontologies may comprise different levels, wherein preferably different levels are associated to different generalizations of metabolite measurements (M), wherein the level of generalization relates to chemical and/or biochemical characteristics of the metabolites.

In a further embodiment the ontology is based on a chemical or biochemical generalization of metabolites. In a further embodiment the metabolite measurements are assigned to at least two hierarchy levels of ontologies (F1, F2), preferably wherein the first ontology level is defined according to a biomolecular or bio-functional classification of metabolites; more preferably wherein the second ontology level is defined according to biochemical relation of metabolites.

Typically, a first ontology level is defined that assigns the metabolites to biomolecular classes, such as amino acids, nucleobases, carbohydrates, lipids, steroids, or terpenes; to chemical classes, such as organic acids; or to classes of biochemical function, e.g. phytohormones, antioxidants, or cofactors.

A second ontology level may be defined that further sub-classifies the assignments in the first ontology level based on groups of biochemically related classes of compounds. Biochemical relation may be established by involvement of a compound in biochemical pathways, such as metabolic pathways (e.g. glycolysis, gluconeogenesis, citric cycle, urea cycle, amino acid synthesis, shikimate pathway, fatty acid and fatty alcohol synthesis), in biosynthesis pathways (e.g. of terpenes, terpenoids and sesquiterpenes, phenylpropanoids, secondary metabolites, components of the cell wall or organelles), redox-pathways (e.g. photorespiration, and redox-equivalents), or by classes of carbohydrates and their derivatives (e.g. mono- and oligosaccharides, sugar acids, sugar alcohols, sugar phosphates).

New metabolite features (Mn) may be derived from the first level of ontologies by determining the average of received metabolite measurements (M) for all metabolites that have been assigned to the same ontology. Accordingly, the classified average may be determined in a two-step process, wherein in a first step the received metabolite measurements (M) are assigned to at least one ontology (F1, F2), and wherein in a second step the average of metabolite measurements (M) is determined for those received metabolite measurements (M) that are assigned to the same ontology.

A third ontology level may be defined. In this third ontology, metabolites are first assigned to the same class of metabolites if they are either a substrate or a product of the same enzyme. Enzymes may be identified by their “Enzyme Classification Number”. For example, the fumarate hydratase (EC number 4.2.1.2) transforms fumarate to malate in the citrate cycle. Accordingly, fumarate and malate would be assigned to the same ontology class of level three. In a second step, the ratio is calculated between the amount of the product(s) to the substrate(s) within this class, e.g. the ratio of fumarate to malate. In one embodiment of the invention additional new metabolite features (Mn) are determined in a process including a first step of assigning the received metabolite measurements (M) to different ontologies (F3) based on a classification of metabolites as substrate(s) or product(s) of an enzymatically catalyzed reaction; and a second step of determining a ratio between product metabolite measurements and substrate metabolite measurements of the same ontology.

While some enzymes only catalyze the reaction of a given substrate to a product (forward reaction), many enzymes catalyze both the forward reaction and the backward reaction from the product of the forward reaction to the substrate of the forward reaction. Such enzymes typically lower the kinetic energy to reach the equilibrium state in which the Gibbs free energy reaches a minimum. This is for example the case for most isomerases and epimerases.

Accordingly, the determination of the ratio between product metabolite measurements and substrate metabolite measurements may relate to the forward reaction or the backward reaction with regard to the identity of a metabolite as product or substrate of the enzymatically catalyzed reaction.

In a further embodiment new metabolite features (Mn) are determined by the steps of:

a) assigning the received metabolite measurements (M) to different ontologies based on a classification of metabolites as substrate(s) or product(s) of an enzymatically catalyzed reaction; and

b) determining a ratio between product metabolite measurements and substrate metabolite measurements.

In a further embodiment the received metabolite measurements and the new metabolic features are provided to the trained machine learning model. This way the model is fed with the non-averaged as well as the averaged features making the prediction more robust. Additionally the impact of the averaged as well as non-averaged features can be analyzed to select only those having an impact on the model prediction and its quality. This way the number of measurements needed to use the method can be reduced.

In a further embodiment the model is a classification model. In a further embodiment the model is an ensemble classification model combining several classification models. The ensemble classification model may be based on a voting classifier combining more than one model classifier using a majority or averaged probability. This is particularly advantageous for yield performance predictions, where the historical data is typically limited, and robustness of the prediction is key. Classification models of different types such as tree-based boosting or averaging models may be used in this context.

The model preferably is a machine learning model being trained on a training dataset containing the measured and/or new metabolite features. Training datasets are used to train the model. Test datasets are used to test the model performance. The machine learning model predicts the yield performance e.g. whether a crop plant will have a yield loss or not.

The machine learning algorithm preferably comprises decision trees, naive bayes classifications, nearest neighbors, neural networks, convolutional neural networks, generative adversarial networks, support vector machines, linear regression, logistic regression, random forest and/or gradient boosting algorithms.

The machine learning algorithm preferably is carried out by a artificial intelligence module. The artificial intelligence module is an entity that processes one or more inputs into one or more outputs by means of an internal processing chain that typically has a set of free parameters. The internal processing chain may be organized in interconnected layers that are traversed consecutively when proceeding from the input to the output.

Many artificial intelligence modules are organized to process an input having a high dimensionality into an output of a much lower dimensionality. Such a module is termed “intelligent” because it is capable of being “trained.” The module may be trained using records of training data. A record of training data comprises multiple training input data sets and corresponding training output data sets. The training output data of a record of training data is the result that is expected to be produced by the module when being given the training input data of the same record of training data as input. The deviation between this expected result and the actual result produced by the module is observed and rated by means of a “loss function”. This loss function is used as a feedback for adjusting the parameters of the internal processing chain of the module. For example, the parameters may be adjusted with the optimization goal of minimizing the values of the loss function that result when all training input data is fed into the module and the outcome is compared with the corresponding training output data. The result of this training is that given a relatively small number of records of training data as “ground truth”, the module is enabled to perform its job well for a number of records of input data that is higher by many orders of magnitude.

The model is preferably trained and tested on measured variables coming from different growth stages, such as the reproductive growth stage and/or vegetative growth stage of the crop plant. Preferably, at least one crop cycle, in particular one year, is excluded from the training of the model in order to test the model. Preferably, the model is trained and tested to predict two classes of yield: “yield loss” or “no yield loss”.

In a preferred embodiment, the model determines the yield performance for the running or the next crop cycle, in particular the next year. This step is repeated each time for training and testing the vegetative data of the excluded year to understand the model performance and the impact of individual input dimensions on the prediction performance.

In a preferred embodiment, the accuracy of the model is higher than 75%, further preferably in range of 79% to 86%, 70% to 99% or 75% to 90%.

Thus, the search space for identifying the most relevant biomarkers, in particular new metabolite features, can be reduced. This is particularly advantageous for use of the model. Once the most relevant biomarkers impacting the prediction performance of the model are identified, the model can be trained on a reduced set of input dimensions, e.g. based on such identified biomarkers. Therefore, the yield performance prediction of the crop plant can be improved and a link between a crop plant response in the field and in the greenhouse can be established.

In a further embodiment, a pre-training process is conducted to identify the minimum input dimensions required by the model. In such an embodiment the model is trained based on historical data sets of measured metabolites and corresponding new metabolite features, the model performance is tested during training and the most relevant measured metabolites and corresponding new metabolite features are identified. In this context most relevant refers to measured metabolites and corresponding new metabolite features with the highest prediction power or most impact on an accurate prediction. Based on the identified measured metabolites and corresponding new metabolite features the historical data sets may be pruned and the model training can be conducted based on the pruned historical data sets. This way the input dimensions required for the model to make predictions can be reduce to a minimum set. This has the advantage of reducing the number of measurements required to be received to use the model in the method for predicting yield performance of a crop plant.

Thus, the plant performance is improved by improving precision and efficiency in plant breeding, crop trait development for crops as well as development of biostimulants. It further helps farming practice in farmers' decisions such as irrigation.

Preferably, the described method for predicting yield performance of a crop plant comprises the steps:
receiving hyperspectral data of the crop plant;
determining vegetation indices, relating to a combination of spectral bands from the crop plant, preferably having physiological meaning, from the hyperspectral data; and
providing the determined vegetation indices to the trained machine learning model.

In a preferred embodiment, in addition to the hyperspectral data of the crop plant, thermography data of the crop plant as well as data about crop plant volume and/or plant height are received and used by the machine learning algorithm to determine the model.

In a preferred embodiment the hyperspectral data is gathered by spectral imaging with visible and near-infrared (VNIR) and/or short wavelength infrared (SWIR), the thermography data is gathered by thermography imaging.

Preferably, the hyperspectral data is provided by a hyperspectral sensor, like a type of camera, that flies over the crop field in an aeroplane or a drone. The hyperspectral sensor preferably produces images where every pixel has full spectral information and the data are retrieved in spectral bands, also called wavelengths. The hyperspectral data preferably is a 3D data set, where first and second dimensions are the surface of the object and on top every layer is the information in one spectral band at a time. Once the wavelength is acquired, it is used to calculate vegetation indices.

Thus, the prediction of the yield performance of the crop plant can be further improved and a link between a crop plant response in the field and in the greenhouse can be established.

Preferably, the described method comprises the steps:

determining the metabolite measurements based on a crop plant sample by chromatography, preferably polar gas chromatography, lipid gas chromatography, polar liquid chromatography and/or lipid liquid chromatography.

Preferably, the historical data sets for training include hyperspectral data and/or metabolite measurements from crop field trials with different levels of abiotic stress, preferably drought stress.

The term “crop field trial”, as used herein, comprises the cultivation of different variations of crop plants (e.g. genetic variations) of the same crop plant type (e.g. of the same crop plant variety) under predetermined conditions, in particular a greenhouse and/or on the crop field, in order to evaluate the performance of the different variations of crop plants, in particular for breeding purposes, trait selection purposes and/or biostimulant treatment selection purposes.

In order to develop a method for predicting yield performance of a plant, a common parameter has to be found that is measurable at all stages of plant growth, the vegetative growth stage in the greenhouse and/or the crop field and the reproductive growth stage in the crop field.

Preferably, biomarkers, in particular new metabolite features, are identified to build a link between vegetative and reproductive growth stages within the crop plant in the greenhouse and the field that account for the abiotic stress, in particular the drought stress, and mainly yield impact. To find these potential biomarkers based on crop field trials, preferably at least two simultaneous crop field trials are set up using a randomized block design with at least three different levels of abiotic stress, preferably with a level of no abiotic stress as control. Preferably, the plants are subjected to these different treatment levels at vegetative or reproductive growth stages respectively.

The crop field trials are preferably conducted over at least two years, further preferably at least two crop cycles. Preferably, within one crop cycle, crop plant samples of the crop field trials of several, preferably at least three, time points are analyzed. In a preferred embodiment, the crop field trials comprise at least two different varieties of plant seeds, preferably corn seeds. Preferably, at least one common variety of plant seeds is used throughout the crop field trials. In other words, one common variety of plant seeds is used over several, in particular all, crop cycles. At the end of the crop cycle, the actual yield per plant in the plant crop field stressed at all stages of plant growth, the vegetative growth stage and the reproductive growth stage, is measured.

Preferably, the crop plant is cultivated in the greenhouse for the vegetative growth stage, in particular early stages of the vegetative growth stage, in particular up to BBCH stage 31.

Preferably, the vegetative growth stage in the greenhouse relates to a BBCH stage of the crop plant of 10, in particular relating to one leaf, up to 31, in particular relating to one node. Further preferably, the vegetative growth stage in the field relates to a BBCH stage of the crop plant of 15 up to 30-32. Further preferably, the reproductive growth stage of the crop plant relates to a BBCH stage of the crop plant from 30-32 up to 67.

Preferably, drought stress leads to common crop plant responses in the crop field and in the greenhouse. Thus, an improved link between the greenhouse and the crop field can be established.

Thus, a link between a crop plant response, in particular a yield performance caused by drought stress, between a crop plant in the vegetative growth stage in the greenhouse and a crop plant in the vegetative growth stage in the field, as well as between a crop plant in the vegetative growth stage in the field and a crop plant in the reproductive growth stage in the field can be established.

Thus, the prediction of the yield performance of the crop plant can be further improved and a link between a crop plant response in the field and in the greenhouse can be established.

In a preferred embodiment for training the model, the crop field trials are performed during a vegetative and/or a reproductive growth stage of the plant.

Preferably, the described method comprises the steps:

determining the new metabolite features combining the received metabolite measurements based on assignments of the received metabolite measurements to different ontologies and/or based on a ratio between product metabolites and substrate metabolites.

Preferably, the ontologies comprise different levels, wherein preferably different levels are associated to different generalizations of metabolite measurements.

Therefore, the metabolite measurements can be further grouped.

Thus, the prediction of the yield performance of the crop plant can thus be further improved and a link between a crop plant response in the field and in the greenhouse can be established.

In a preferred embodiment, a first level of ontologies preferably comprises one of amino acids and related or organic acids or carbohydrates and related or complex lipids, fatty acids and related or secondary metabolism or phytohormones or nucleobases and related or miscellaneous. A second level of ontologies as an example of a subgroup of carbohydrates and related comprises sugar alcohol or free sugars or sugar phosphates or sugar acids.

Preferably, the described method comprises the steps:

validating the yield performance prediction data and providing validation data by comparing the yield performance prediction data with the actual yield data of the respective crop plant; and

adjusting the model based on the validation data.

Preferably, the described method comprises the steps:

adjusting a parametrization of a machine learning algorithm determining the model based on the validation data; wherein the machine learning algorithm comprises several combined machine learning algorithms, in particular three combined machine learning algorithms.

Thus, the prediction of the yield performance of the crop plant can be further improved and a link between a crop plant response in the field and in the greenhouse can be established.

Preferably, the described method comprises the steps:

determining a best metabolite feature from the metabolite feature based on the validation data; wherein the best metabolite feature comprises the metabolite measurements with the highest impact on the expected yield performance; and wherein preferably the best metabolite feature comprises metabolite measurements extracted by polar gas chromatography.

In a preferred embodiment, the machine learning algorithm is only trained by metabolite measurements, which already have been proven to be especially relevant to qualify as a good predicting biomarker.

In a preferred embodiment, the yield performance prediction data preferably comprises the states “yield performance” and “no yield performance”.

Preferably, the yield performance prediction data is determined within the vegetative growth stage of the plant, preferably when the crop plant is still cultivated within a greenhouse.

Thus, a potential yield performance due to abiotic stress of the analyzed variation of crop plant can already be predicted within the vegetative growth stage. Additionally, the potential yield performance can already be predicted, when the crop plant is still in the greenhouse and thus before the crop plant is transferred to the crop field for further cultivation. Therefore, a lot of time and expenses can be saved, especially since the costs for cultivating the crop plant increases by time and the costs for cultivating the crop plant in the greenhouse are significant less than the costs for cultivating the crop plant in the field. Crop plants with high predicted yield performance due to abiotic stress therefore can be sorted out earlier.

A further aspect relates to a control unit being configured for executing a method, described herein.

The control unit may refer to a data processing element such as a microprocessor, microcontroller, crop field programmable gate array (FPGA), central processing unit (CPU), digital signal processor (DSP) capable of receiving crop field data, e.g. via a universal service bus (USB), a physical cable, Bluetooth, or another form of data connection.

A further aspect relates to a yield evaluation platform, comprising optionally a hyperspectral sensor configured for determining hyperspectral data of a plant, a profiling platform configured for determining metabolite measurements from a crop plant sample and a control unit, as described herein.

A further aspect relates to a plant breeding method, comprising the steps:

optionally cultivating crop plants;

determining yield performance prediction data of the respective or more than one crop plant using the method for predicting yield performance, as described herein; and

selecting the crop plants according to a predicted yield performance, e.g. a yield loss below a predetermined threshold for future breeding cycles.

In a preferred embodiment, the plant breeding method is not limited to crop plants, but any kind of germplasm.

Therefore, a breeder can select the crop plants with the highest predicted yield potential for further breeding in an improved way.

In a preferred embodiment, a trait selecting method is provided, comprising the steps: optionally cultivating crop plants;

determining yield performance prediction data of the respective crop plant using the method for predicting yield performance, as described herein; and

selecting the trait with a predicted yield performance for the crop plant, e.g. according to a predetermined threshold.

Therefore, the trait with the highest predicted yield potential for the crop plant can be selected in an improved way.

In a preferred embodiment, a biostimulant treatment selecting method is provided, comprising the steps:

optionally cultivating crop plants that are treated with a biostimulant, and preferably additionally without treatment with a biostimulant;

determining yield performance prediction data of the respective crop plant that have been treated with a biostimulant, and preferably additionally without treatment with a biostimulant, using the method for predicting yield performance, as described herein; and

selecting the biostimulant treatment with a predicted yield performance for the crop plant according to the yield performance (e.g. below a predetermined threshold).

The term “biostimulants” comprises substances and/or microorganisms applied to crop plants that stimulate natural processes in the plant to enhance nutrient uptake or nutrient efficiency, improve tolerance to abiotic stress or crop quality. In agriculture, biostimulation is complementary to crop plant nutrition, for example using fertilizers and crop protection, being products to control pathogens, pests and/or weeds.

Therefore, the biostimulant treatment with the highest predicted yield potential for the crop plant can be selected in an improved way.

A further aspect relates to a farming method, comprising the steps:

determining yield performance prediction data of the respective crop plant using the method for predicting yield performance, as described herein;

providing an expected yield performance of the crop plant depending on the determined yield performance, e.g. to farmers; and

adjusting the farming conditions by the farmer depending on the expected yield performance of the crop plant.

Therefore, a farmer can adjust the farming conditions of the respective field, in order to prevent unwanted yield performance.

Preferably, the farmer cultivates the crop plant for a certain amount of time, for example 15 days. After that, the farmer provides a sample of the crop plant to an external service, for example a vendor. The external service executes the method for yield performance prediction, as described herein, preferably by a yield evaluation platform, as described herein, on the provided sample of the crop plant. The external service then provides the farmer with a predicted yield performance based on yield performance prediction data determined by the method for yield performance prediction. The farmer then adjusts the farming condition on his field depending on the predicted yield performance, for example by increasing the watering amount and/or using yield enhancing products.

A further aspect relates to a use of new metabolite features combining received metabolite measurements of a crop plant sample for yield performance prediction of the crop plant.

Preferably, the use of new metabolite features combining received metabolites of a crop plant sample for yield performance prediction of the plant is based on assignments of the received metabolite measurements to different ontologies and/or based on a ratio between product metabolite measurements and substrate metabolite measurements as described herein.

In a preferred embodiment, a computer program is preferably provided that when it is executed on a control unit, as described herein, instructs the control unit to execute steps of a method, as described herein.

In a preferred embodiment, a computer readable storage medium is preferably provided, being configured to store a computer program, as described herein.

In a preferred embodiment, a crop field management system is provided, being configured for detection of a crop failure. Preferably, the crop field management system is provided with yield performance prediction data by the control unit and is configured for determining the crop failure if the predicted yield performance does exceed a predetermined crop failure threshold.

The crop field management system is further preferably configured for taking actions counteracting the predicted crop failure, if the crop field management system determines the crop failure. Preferably the actions counteracting the prediction crop failure comprises reducing abiotic stress factors like drought, extreme temperature, UV-radiation and/or nutrient deficiency, and/or applying crop protection products like fungicides, herbicides and/or insecticides and/or applying yield enhancing chemicals, microbes, natural compounds and/or natural extracts.

Thus, the yield of plants with expected low yield/high yield performance, can be improved and a link between a crop plant response in the field and in the greenhouse can be established.

Alternatively, the crop field management system is further preferably configured for removing plant candidates, if the crop field management system determines crop failure. Removing crop plant candidates with predicted desirable yield performance helps reducing the maintenance costs of the crop field.

In a preferred embodiment, a plant breeding management system is provided, being configured for determining yield performance prediction data of the respective crop plants using the method for predicting yield performance, as described herein. The plant breeding management system is further configured for selecting the crop plants with a predicted yield performance according to a predetermined threshold for future breeding cycles.

In a preferred embodiment, the plant breeding management system is not limited to crop plants, but any kind of germplasm.

Preferably, the plant breeding management system is configured for controlling a plant treatment device, which is configured for treating plants. The plant treatment device comprises means for sucking, pulling and/or stamping plants. Preferably, the plant treatment device is mounted on an automated unmanned working machine like a drone.

In a preferred embodiment, the crop field management system is configured for cultivating crop plants, determining yield performance prediction data of the respective crop plant using the method for predicting yield performance, as described herein and selecting the trait with a predicted yield performance for the crop plant below a predetermined threshold.

In a preferred embodiment, the crop field management system is configured cultivating crop plants, determining yield performance prediction data of the respective crop plant using the method for predicting yield performance, as described herein and selecting the biostimulant treatment with a predicted yield performance for the crop plant below a predetermined threshold.

Advantageously, the benefits provided by any of the above aspects equally apply to all of the other aspects and vice versa. The above aspects and examples will become apparent from and be elucidated with reference to the embodiments described hereinafter.

### BRIEF DESCRIPTION OF THE DRAWINGS

Exemplary embodiments will be described in the following with reference to the following drawings:

FIG. 1 shows a schematic diagram of a yield evaluation platform;

FIG. 2 shows a schematic diagram of a metabolite profiling process;

FIG. 3 shows a schematic diagram of the combination of metabolite measurements;

FIG. 4 shows a schematic diagram of a control unit; and

FIG. 5 shows a schematic diagram of a method for predicting yield performance of a crop plant.

### DETAILED DESCRIPTION OF EMBODIMENTS

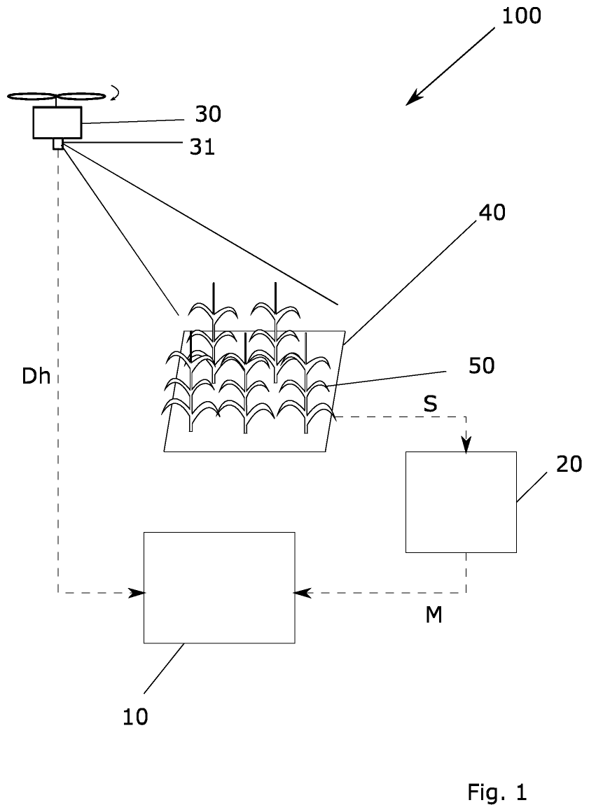

FIG. 1 shows a yield evaluation platform 100 comprising a crop field 40 cultivating crop plants 50. In this example, the crop plants 50 comprise corn plants. In order to make assumptions regarding the expected yield of the crop plants 50, measurable indicators, so called biomarkers, have to be found.

Developing relevant biomarker, different crop field trials have to be run on different setups, in particular on different levels of abiotic stress like drought stress. Therefore, different crop plants 50 or areas of crop plants 50 on the crop field 40 are continuously stressed by constant levels of drought stress. For example two simultaneous field trials are set up using randomized block design with three different levels of water treatment. As a comparison, a control group of crop plants 50, which are not stressed is also added. Crop plants 50 are subjected to these different treatment levels at vegetative or reproductive growth stages, respectively. The same trials are conducted for several subsequent years. The crop plant trails are run from the vegetative growth stage, about thirty days, through the reproductive phase, about sixty days, of the crop plant 50. Ideally, a biomarker can be found that has a high field predictive power and allows assumptions on the yield or the yield performance of a crop plant 50 within the vegetative growth stage. Thus, assumptions on the yield or the yield performance of a crop plant 50 in the field can be made while the crop plant 50 is still in the greenhouse, preferably at an early stage of the vegetative growth stage.

The crop field trials use drought stress as abiotic stress to evaluate different biomarkers on their predictive power regarding the yield performance of the crop plant 50 relating to the drought stress. The expected yield performance of a crop plant 50 increases with the amount of drought stress applied to the crop plant 50.

For each experimental setup in crop field trials, at least two different varieties of crop seeds are used, but at least one common variety is maintained throughout the crop field trials.

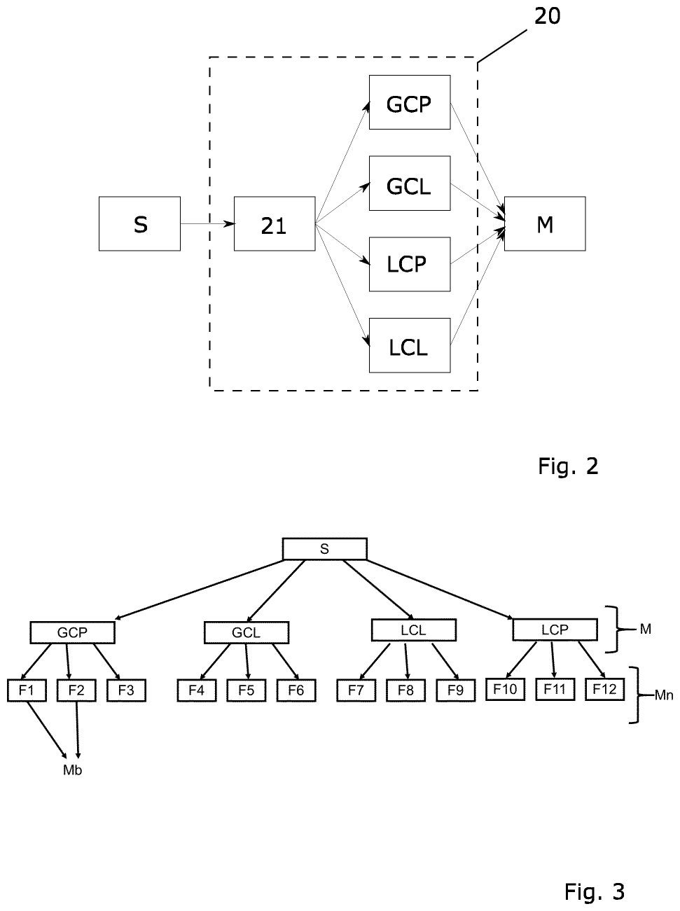

For finding a valid biomarker, on different stages of the crop field trial, crop plant samples S of the different crop plants 50 are taken. For example, the crop plant sample S is corn leaf tissue. For example, crop plant samples S from three different time points are taken. The crop plant samples S are then provided to a profiling platform 20, generating metabolite measurements M from the crop plant samples S and providing them to the control unit 10, as described in detail in FIG. 2.

Additional information about the crop field 40 is gathered by remote sensing. Therefore, a hyperspectral sensor 31, which is preferably mounted on a drone 30, gathers hyperspectral and thermal information, as well as information about the volume and the height of the crop plants 50. The hyperspectral sensor 31 is therefore configured for gathering hyperspectral data Dh, in particular by spectral imaging with visible and near-infrared (VNIR) and/or short wavelength infrared (SWIR). The hyperspectral data Dh, is provided to the control unit 10.

Instead of a drone 30, the hyperspectral sensor 31 can be mounted on any manned or unmanned working machine.

FIG. 2 shows a schematic diagram of a metabolite profiling process using the profiling platform 20. A crop plant sample S is provided to the profiling platform 20. The profiling platform 20 comprises a preparation unit 21, being configured for freeze-drying and/or milling the crop plant sample S and being configured for extraction and separation of the crop plant sample S in lipid and polar phase. One extraction of the crop plant sample S thereby delivers the whole spectrum of metabolite measurements M.

As is shown, from a single crop plant sample S four different data sets can be received. The total number of metabolite measurements M identified with all four data sets is around 750 metabolite measurements M. The metabolite measurements M are determined by polar gas chromatography GCP, lipid gas chromatography GCL, polar liquid chromatography LCP and/or lipid liquid chromatography LCL and then provided to the control unit 10.

FIG. 3 shows a schematic diagram of the combination of metabolite measurements M. The metabolite measurements M are assigned to different ontologies, by which metabolite measurements M are combined.

In this example, the metabolite features Mn determined by polar gas chromatography GCP are partly assigned to a first ontology F1, for example comprising organic acids, amino acids and related and carbohydrates and related.

In this example, the metabolite features Mn determined by polar gas chromatography GCP are partly assigned to a second ontology F2, for example comprising sugar alcohols, sugar phosphates and free sugars.

In this example, the metabolite features Mn determined by polar gas chromatography GCP are defined by different ratios between two metabolite measurements M that is defined as product and substrate in an enzyme mapping F3.

Using this method, new metabolite features Mn are determined combining the received metabolite measurements M. The new metabolite features Mn are then provided to a model 13 of the control unit 10.

FIG. 3 shows that similar assignments are done for lipid gas chromatography GCL, polar liquid chromatography (LCP) and lipid liquid chromatography (LCL) determining fourth to twelfth ontologies F4 to F12.

The different ontologies F1 to F12 are validated and best new metabolites features Mb are determined from the new metabolite features Mn, wherein the best metabolite features Mb comprises the metabolite features Mn with the highest impact on the expected yield performance. In this case the first ontology F1 and the second ontology F2 are the best new metabolite features Mb.

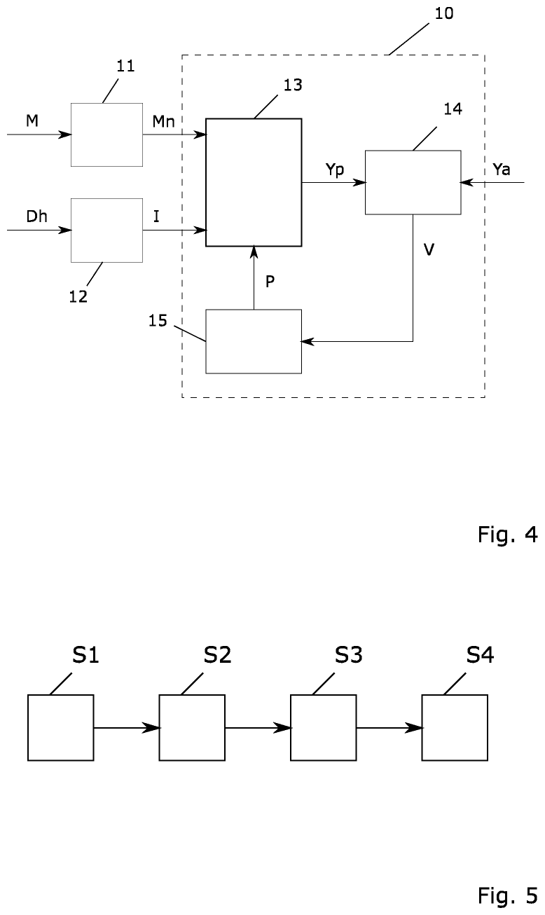

FIG. 4 shows a schematic diagram of the control unit 10, comprising the ontology unit 11, vegetation indices unit 12, the model 13, a yield prediction unit 14 and a machine learning unit 15. As described above, the new metabolite features Mn are provided to the model 13. Additionally, the hyperspectral data Dh are also provided to the vegetation indices unit 12. The vegetation indices unit 12 calculates vegetation indices I. The vegetation indices I are knowledge driven variables as they have physiological meaning in crop plants. The vegetation indices I are then provided to the model 13.

The model 13 is provided with parameters P from the machine learning unit 14. Based on the parameters P and the provided data from the ontology unit 11 and the hyperspectral sensor 31, the model 13 is trained. The model 13 then is used to provide a yield prediction data Yp of the respective crop plant 50. The model is preferably trained and tested to predict two classes of yield: “yield loss” and “no yield loss”. The yield prediction data Yp of the model 13 is provided to the validation unit 14. If available, the validation unit 14 additionally is provided with actual yield data Ya. The validation unit 14 then compares the yield prediction data Yp with the actual yield data Ya and determines validation data V, representing the accuracy of the yield prediction data Yp. The validation data V is provided to the machine learning unit 15, which adjusts the parameters P provided to the model 13 based on the validation data V.

The control unit 10, the ontology unit 11, the vegetation indices unit 12, the model 13, the validation unit 14 and/or the machine learning unit 15 may refer to a data processing element such as a microprocessor, microcontroller, crop field programmable gate array (FPGA), central processing unit (CPU), digital signal processor (DSP) capable of receiving crop field data, e.g. via a universal service bus (USB), a physical cable, Bluetooth, or another form of data connection. The respective units may be several independent devices. However, more or all respective units may be integrated into one device.

FIG. 5 shows a schematic diagram of a method for predicting yield performance of a crop plant 50.

In step S1, metabolite measurements M of the crop plant 50 are received. In step S2, new metabolite features Mn are determined combining the received metabolite measurements M. In step S3, a model 13 is determined by a machine learning algorithm based the new metabolite features Mn. In step S4, yield performance prediction data Yp of the crop plant 50 is determined using the determined model 13.

### REFERENCE SIGNS

10 control unit
11 ontology unit
12 vegetation indices unit
13 model
14 validation unit
15 machine learning unit
20 profiling platform
21 preparation unit
30 drone
31 hyperspectral sensor
40 crop field
50 crop plant
100 yield evaluation platform
S crop plant sample
Yp yield prediction data
Ya actual yield data
P parameter
V validation data
M metabolite feature
Mn new metabolite features
Mb best metabolite features
GCP polar gas chromatography
GCL lipid gas chromatography
LCP polar liquid chromatography
LCL lipid liquid chromatography
F1 first ontology
F2 second ontology
F3 ratio between product metabolites and substrate metabolites
F4 to F12 Fourth to twelfth ontology
Dh hyperspectral data
I indices
receiving metabolite measurements
S2 determining new metabolite features
S3 determining a model
S4 determining yield performance prediction data

1. A method for predicting yield performance of a crop plant, the method comprising:
receiving (S1) metabolite measurements (M) of the crop plant (50);
determining (S2) new metabolite features (Mn) by combining the received metabolite measurements (M), wherein at least one new metabolite feature (Mn) is based on a classified average;
providing (S3) the new metabolite features to a trained machine learning model (13); and
determining (S4) yield performance (Yp) of the crop plant (50) using the provided model (13).

2. The method of claim 1, further comprising:
receiving hyperspectral data (Dh) of the crop plant (50);
determining vegetation indices (I), relating to a combination of spectral bands from the crop plant (50), preferably having physiological meaning, from the hyperspectral data (Dh); and
providing the vegetation indices to the trained machine learning model (13).

3. The method of claim 1, further comprising:
determining the metabolite measurements (M) based on a crop plant sample (S) by chromatography, preferably polar gas chromatography (GCP), lipid gas chromatography (GCL), polar liquid chromatography (LCP) and/or lipid liquid chromatography (LCL).

4. The method of claim 1, wherein the classified average is determined by
a) assigning the received metabolite measurements (M) to at least one ontology (F1, F2); and
b) determining the average of the metabolite measurements (M) that are assigned to the same ontology.

5. The method of claim 4, wherein the ontology includes metabolite measurements (M) at different points in time during the crop cycle.

6. The method of claim 4, wherein the ontology is based on a chemical or biochemical generalization of metabolites.

7. The method of claim 4, wherein the metabolite measurements (M) are assigned to at least two hierarchy levels of ontologies (F1, F2), preferably wherein the first ontology level is defined according to a biomolecular or bio-functional classification of metabolites; more preferably wherein the second ontology level is defined according to biochemical relation of metabolites.

8. The method of claim 1, wherein new metabolite features (Mn) are determined by
a) assigning the received metabolite measurements (M) to different ontologies (F3) based on a classification of metabolites as substrate(s) or product(s) of an enzymatically catalyzed reaction; and
b) determining a ratio between product metabolite measurements and substrate metabolite measurements.

9. The method of claim 1, wherein the received metabolite measurements (M) and the new metabolic features (Mn) are provided to the trained machine learning model (13).

10. The method of claim 1, wherein the yield performance (Yp) is determined based on metabolite measurements from the vegetative and/or reproductive growth stage of the crop plant (50).

11. A method for training a machine learning model for predicting yield performance of a crop plant, the method comprising:
receiving historical data sets comprising metabolite measurements in connection with a measured yield performance, wherein each data set comprises metabolite measurements for different points in time of the growth cycle for one or more crop plant(s);
determining new metabolite features combining the received historical data sets, wherein at least one new metabolite feature is based on a classified average;
generating a training data set and a test data set based on the historical data sets with new metabolite features;
providing a machine learning model and training the machine learning model based on the training data set; and
testing the trained machine learning model based on the test data set.

12. The method of claim 11, further comprising:
on training, validating the yield performance (Yp) and providing validation data (V) by comparing the predicted yield performance (Yp) with the actual yield performance (Ya) of the respective crop plant (50); and
adjusting the model (13) based on the validation data (V).

13. The method of any of claim 11, further comprising:
adjusting a parametrization (P) of a machine learning algorithm determining the model (13) based on the validation data (V).

14. The method of claim 11, further comprising:
determining a best new metabolite feature (Mb) from the new metabolite features (Mn) based on the validation data (V); wherein
the best metabolite feature (Mb) comprises the metabolite measurements (M) with the highest impact on the expected yield performance; and
wherein preferably the best metabolite feature (Mb) comprises metabolite measurements (M) extracted by polar gas chromatography (GCP).

15. A control unit (10) being configured for executing the method of claim 1.

16. A yield evaluation platform (100), comprising:
a profiling platform (20) configured for determining metabolite measurements (M) from a crop plant sample (S); and
a control unit (10) of claim 15.

17. A plant breeding method, comprising:
determining yield performance (Yp) per plant of more than one crop plant (50) using the method of claim 1; and
selecting the crop plants (50) with a predicted yield performance (Yp) according to predicted yield performance (Yp) for future breeding cycles.

18. A farming method, comprising:
determining yield performance (Yp) of one or more crop plant(s) (50) using the method of claim 1;
providing an expected yield performance of the crop plant(s) (50) depending on the determined yield performance (Yp); and
adjusting farming conditions by the farmer depending on the expected yield performance of the crop plant(s) (50).

19. (canceled)
